(19) United States
(12) Patent Application Publication
(10) Pub. No.: US 2022/0279787 A1
(43) Pub. Date: Sep. 8, 2022

(54) a-Substituted Phenyl Structure-Containing Compound, Preparation Method Thereof, and Disinfectant

(71) Applicant: Henan Ruibo Pharmaceutical Technology Co. Ltd., Zhengzhou (CN)

(72) Inventors: Hongmin Liu, Zhengzhou (CN); Bing Zhao, Zhengzhou (CN); Shangshang Qin, Zhengzhou (CN); Liying Ma, Zhengzhou (CN); Kai Sun, Zhengzhou (CN); Xuejian Feng, Zhengzhou (CN); Wenge Guo, Zhengzhou (CN); Guangyao Liu, Zhengzhou (CN)

(21) Appl. No.: 17/632,004

(22) PCT Filed: Jun. 24, 2020
(86) PCT No.: PCT/CN2020/098049

§ 371 Date: Feb. 1, 2022

(30) Foreign Application Priority Data
Aug. 6, 2019 (CN) 201910720391.7

(51) Int. Cl. A01N 43/40; C07C 235/34; C07C 233/11; C07C 59/52; C07D 307/54; C07D 213/56; A01N 37/18; A01N 37/10; A01N 43/08; A01P 1/00

(52) U.S. Cl. CPC A01N 43/40; C07C 235/34; C07C 233/11; C07C 59/52; C07D 307/54; C07D 213/56; A01N 37/18; A01N 37/10; A01N 43/08; A01P 1/00

(57) ABSTRACT

The disclosure relates to the technical field of sterilizing and disinfecting materials, and specifically relates to an α-substituted phenyl structure-containing compound, a preparation method thereof, and a disinfectant. The α-substituted phenyl structure-containing compound according to the disclosure could achieve a bactericidal effect by promoting the coagulation and denaturation of the protein of pathogenic microorganisms. In particular, it has a good killing effect on pathogenic bacteria such as Escherichia coli, Staphylococcus aureus, Pseudomonas aeruginosa, and Bacillus subtilis var. niger spores. Besides, it has no corrosive effect on metals, no irritating odor, good water solubility, and is green and environmentally friendly. Therefore, it could be widely used in various industries as an effective ingredient of a disinfectant.

### CROSS REFERENCE TO RELATED APPLICATION

This application claims the benefit and priority of Chinese Patent Application No. 201910720391.7 filed with the China National Intellectual Property Administration on Aug. 6, 2019, entitled by “α-substituted phenyl structure-containing compound, preparation method thereof, and disinfectant”, the disclosure of which is incorporated by reference herein in its entirety as part of the present application.

### TECHNICAL FIELD

The present disclosure relates to the technical field of sterilizing and disinfecting materials, in particular to an α-substituted phenyl structure-containing compound, a preparation method thereof, and a disinfectant.

### BACKGROUND ART

Disinfectants are widely used in the fields of medical treatment, animal husbandry, forestry and aquaculture. With the increasing requirement for sanitation, the demand for disinfectants is also increasing. The disinfectants currently sold and used in the market can be roughly divided into nine categories: chlorine-containing disinfectants, peroxide disinfectants, ethylene oxide disinfectants, aldehyde disinfectants, and phenolic disinfectants, but they generally have shortcomings such as big taste and undesired bactericidal effect.

### SUMMARY

An object of the present disclosure is to provide an α-substituted phenyl structure-containing compound, a preparation method thereof, and a disinfectant. The disinfectant according to the present disclosure has a good killing effect on pathogenic bacteria such as Escherichia coli, Staphylococcus aureus, Pseudomonas aeruginosa, and Bacillus subtilis var. niger spores, has no pungent odor, and is environmentally friendly.

In order to achieve the above object of the present disclosure, the present disclosure provides the following technical solutions:

Disclosed is an α-substituted phenyl structure-containing compound, which has a structure represented by formula I,

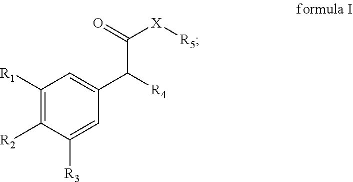

in formula I, each of R₁, R₂ and R₃ is independently selected from the group consisting of hydrogen, hydroxy, fluorine, and methoxy;

R₄ is selected from the group consisting of phenyl, 2-methylphenyl, 3-methylphenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2,3-dihydroxyphenyl, 2-bromophenyl, 3-bromophenyl, 4-bromophenyl, 2-fluorophenyl, 3-fluorophenyl, 4-fluorophenyl, 2,3-dimethylphenyl, 3,4-dimethylphenyl, 3,4,5-trimethylphenyl, 3,4,5-trimethoxyphenyl, 3,4,5-trihydroxyphenyl, pyridyl, 2-methylpyridyl, 3-methylpyridyl, cyclohexyl, furyl, and pyrrolyl;

R₅ is selected from the group consisting of hydrogen, methyl, ethyl, isopropyl, phenyl, and benzyl; and

X is selected from the group consisting of —CH₂—, —NH—, —O—, and —S—.

In some embodiments, the α-substituted phenyl structure-containing compound is one selected from the group consisting of

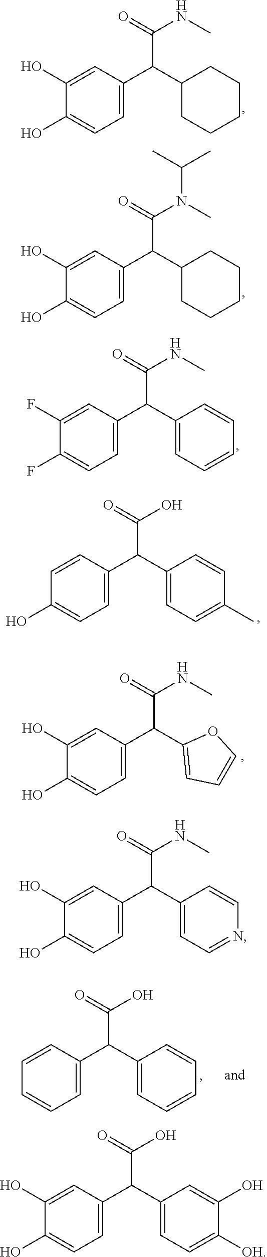

The present disclosure provides a method for preparing the α-substituted phenyl structure-containing compound described in the above technical solutions; under the condition that R₅—X— is a hydroxyl group, the method for preparing the α-substituted phenyl structure-containing compound includes the following steps:

mixing

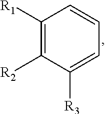

R₄—H, glyoxylic acid, and a catalyst I to undergo a Friedel-Crafts reaction to obtain a compound having a structure represented by formula I;

under the condition that R₅—X— is a group other than hydroxyl, the method for preparing the α-substituted phenyl structure-containing compound includes the following steps: mixing

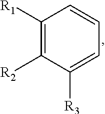

R₄—H, glyoxylic acid and a catalyst I to undergo a Friedel-Crafts reaction to obtain a compound having a structure represented by formula II; and

mixing the compound having the structure represented by formula II, R₅—X—H and a catalyst II to undergo a condensation reaction to obtain a compound having the structure represented by formula I;

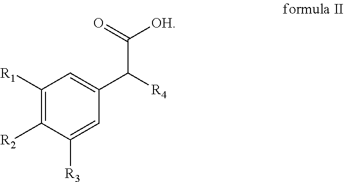

In some embodiments, a molar ratio of

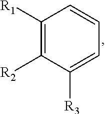

R₄—H, and glyoxylic acid is in the range of 1:(1.1-1.3):(1.3-1.5). In some embodiments, the catalyst I is a strong acid, wherein the strong acid is sulfuric acid or nitric acid.

In some embodiments, the Friedel-Crafts reaction is performed at a temperature of 60-110° C. for 4-8 h.

In some embodiments, the catalyst II is a mixture of 2-(7-azabenzotriazole-yl)-N,N,N′,N′-tetramethyluronium hexafluorophosphate and N,N-diisopropylethylamine. In some embodiments, a molar ratio of the 2-(7-azabenzotriazole-yl)-N,N,N′,N′-tetramethyluronium hexafluorophosphate to the N,N-diisopropylethylamine is in the range of 1.1:(4-10).

In some embodiments, a molar ratio of the compound having a structure represented by formula II, R₅—X—H, and the catalyst II is in the range of 1:1.1:(3-7.1).

In some embodiments, the condensation reaction is performed at ambient temperature for 2-5 h.

The present disclosure also provides a disinfectant, an active ingredient of which includes the α-substituted phenyl structure-containing compound described in the above technical solutions.

The present disclosure provides an α-substituted phenyl structure-containing compound. The compound having the structure represented by formula I could achieve a bactericidal effect by promoting the coagulation and denaturation of protein(s) of pathogenic microorganisms, or by inhibiting the activity of bacterial oxidase, dehydrogenase, catalytic enzyme and other enzymes. In particular, it has a good killing effect on pathogenic bacteria such as Escherichia coli, Staphylococcus aureus, Pseudomonas aeruginosa and Bacillus subtilis var. niger spores. Besides, it has no corrosive effect on metals, no irritating odor, and good water solubility, and is green and environmentally friendly. Therefore, it could be widely used in various industries as an effective ingredient of a disinfectant. It can be seen from the test results of the examples that the α-substituted phenyl structure-containing compound according to the present disclosure has good water solubility, and has a solubility reaching 15 g/L; given a concentration of 3.125 g/L, it has a killing logarithmic value against Escherichia coli ATCC 25922 within 1 min of not less than 5.00, a killing logarithmic value against Pseudomonas aeruginosa ATCC 27853 within 15 min of not less than 5.00; given a concentration of 6.25 g/L, it has a killing logarithmic value against Staphylococcus aureus ATCC 29213 within 15 min of not less than 5.00; given a concentration of 5 g/L, it has a killing logarithmic value against Bacillus subtilis var. niger spores within 10 min of not less than 5.00. Also, it is non-corrosive to metals, and meets the sanitary requirements for phenolic disinfectants according to GB27947-2011 and the sanitary requirements for medical device disinfectants according to GB/T27949-2011.

The method for preparing the α-substituted phenyl structure-containing compound according to the present disclosure is simple and suitable for large-scale production.

### DETAILED DESCRIPTION OF THE EMBODIMENTS

The present disclosure provides an α-substituted phenyl structure-containing compound, which has a structure represented by formula I,

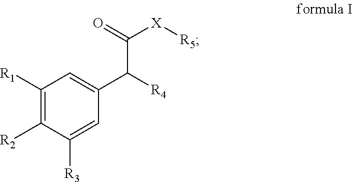

in formula I, each of R₁, R₂ and R₃ is independently selected from the group consisting of hydrogen, hydroxy, fluorine, and methoxy;

R₄ is selected from the group consisting of phenyl, 2-methylphenyl, 3-methylphenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2,3-dihydroxyphenyl, 2-bromophenyl, 3-bromophenyl, 4-bromophenyl, 2-fluorophenyl, 3-fluorophenyl, 4-fluorophenyl, 2,3-dimethylphenyl, 3,4-dimethylphenyl, 3,4,5-trimethylphenyl, 3,4,5-trimethoxyphenyl, 3,4,5-trihydroxyphenyl, pyridyl, 2-methylpyridyl, 3-methylpyridyl, cyclohexyl, furyl, and pyrrolyl;

R₅ is selected from the group consisting of hydrogen, methyl, ethyl, isopropyl, phenyl, and benzyl; and

X is selected from the group consisting of —CH₂—, —NH—, —O—, and —S—.

In some embodiments of the present disclosure, the α-substituted phenyl structure-containing compound includes one selected from the group consisting of

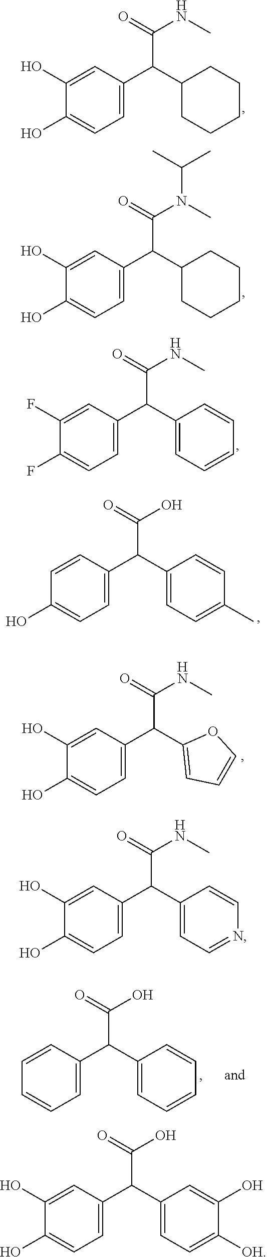

The present disclosure provides a method for preparing the α-substituted phenyl structure-containing compound described in the above technical solutions; under the condition that R₅—X— is a hydroxyl group, the method for preparing the α-substituted phenyl structure-containing compound includes the following steps:

mixing

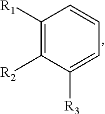

R₄—H, glyoxylic acid and a catalyst I to undergo a Friedel-Crafts reaction to obtain a compound having a structure represented by formula I;

under the condition that R₅—X— is a group other than hydroxyl, the method for preparing the α-substituted phenyl structure-containing compound includes the following steps:

mixing

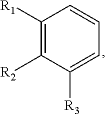

R₄—H, glyoxylic acid, and a catalyst I to undergo a Friedel-Crafts reaction to obtain a compound having a structure represented by formula II; and

mixing the compound having the structure represented by formula II, R₅—X—H, and a catalyst II to undergo a condensation reaction to obtain a compound having the structure represented by formula I;

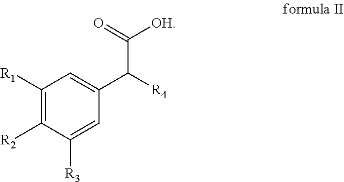

In the present disclosure, unless otherwise specified, all raw materials are commercially available products well known to those skilled in the art.

In the present disclosure, under the condition that R₅—X— is a hydroxyl group, the method for preparing the α-substituted phenyl structure-containing compound comprises the following steps:

mixing

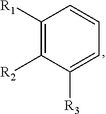

R₄—H, glyoxylic acid, and the catalyst I to undergo a Friedel-Crafts reaction to obtain a compound having the structure represented by formula I.

In the present disclosure, the R₁, R₂, and R₃ in

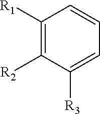

are consistent with R₁, R₂, and R₃ in the structure represented by formula I, and R₄ in R₄—H is consistent with R₄ in the structure represented by formula I, and they will not be repeated here. In some embodiments, the glyoxylic acid is glyoxylic acid monohydrate. In some embodiments, the catalyst I is a strong acid. In some embodiments, the strong acid is sulfuric acid or nitric acid. In a specific embodiment of the present disclosure, a solid strong acid is used as the catalyst I, which is beneficial to the post-treatment.

In some embodiments of the present disclosure, a molar ratio of

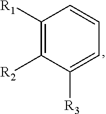

R₄—H, and glyoxylic acid is in the range of 1:(1.1-1.3):(1.3-1.5), and preferably 1:1.1:1.3.

In some embodiments, a molar ratio of

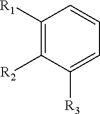

to the catalyst I is in the range of 1:(0.03-0.05), and preferably 1:0.03.

In some embodiments of the present disclosure, the mixing is carried out in water. In some embodiments of the present disclosure, water is used as a solvent, which is more environmentally friendly. In some embodiments of the present disclosure, the water is distilled water. In some embodiments, a ratio of

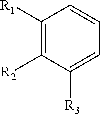

to water is in the range of 1 g:(10-40) mL, and preferably 1 g:20 mL.

In some embodiments of the present disclosure, part of

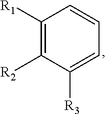

R₄—H, glyoxylic acid, and the catalyst I are first mixed, and the remaining

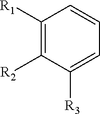

is then added thereto, which is beneficial to the control of the reaction and the completion of the reaction. In some embodiments of the present disclosure, the part of

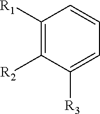

accounts for 50% of the total mass of

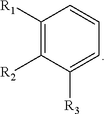

In some embodiments of the present disclosure, the mixing is carried out under a stirring condition. In some embodiments, the stirring is performed at a stirring speed of 160-180 r/min, and preferably 180 r/min.

In some embodiments of the present disclosure, the Friedel-Crafts reaction is performed at a temperature of 60-110° C., and preferably 70-80° C. In some embodiments of the present disclosure, the progress of the Friedel-Crafts reaction is tracked by TLC (thin-layer chromatography) to determine the end time of the reaction. In some embodiments of the present disclosure, the Friedel-Crafts reaction is performed for 4-8 h, and preferably 5-7 h. The time for Friedel-Crafts reaction is specifically started counting after the completion of the addition of the catalyst I.

In some embodiments, after the Friedel-Crafts reaction, the obtained system is subjected to an extraction and a recrystallization in sequence to obtain the compound having the structure represented by formula I (mode 1); or the system obtained after the Friedel-Crafts reaction is diluted by ethyl acetate and then subjected to a filtration, the solid material obtained by the filtration is dissolved in diethyl ether, and the resulting solution is subjected to an extraction with an aqueous sodium carbonate solution; the aqueous layer obtained by the extraction is acidified to a pH value of 2 with concentrated hydrochloric acid, and then the acidified system was filtered to obtain a compound having the structure represented by formula I (mode 2).

In some embodiments, when the compound having the structure represented by formula I is obtained by adopting mode 1, the extraction comprises cooling the system obtained from the Friedel-Crafts reaction to room temperature, adjusting a pH value of the system to 2, and subjecting the system to an extraction with ethyl acetate to obtain a crude product of compound having the structure represented by formula I. In some embodiments of the present disclosure, the recrystallization comprises recrystallizing a crude product of compound having the structure represented by formula I with ethanol.

In some embodiments, when the compound having the structure represented by formula I is obtained by adopting mode 2, the aqueous sodium carbonate solution has a concentration of 1 mol/L. In some embodiments, the extraction is performed for 3 times.

In the present disclosure, under the condition that R₅—X— is a group other than hydroxyl, the method for preparing the α-substituted phenyl structure-containing compound includes the following steps: mixing

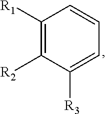

R₄—H, glyoxylic acid, and the catalyst I to undergo a Friedel-Crafts reaction to obtain a compound having a structure represented by formula II; and

mixing the compound having the structure represented by formula II, R₅—X—H, and the catalyst II to undergo a condensation reaction to obtain a compound having the structure represented by formula I.

In the present disclosure,

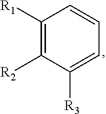

R₄—H, glyoxylic acid, and the catalyst I are mixed to undergo a Friedel-Crafts reaction to obtain a compound having a structure represented by formula II. In the present disclosure, components of

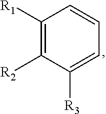

R₄—H, glyoxylic acid, and the catalyst I, amount ratio, mixing process, temperature and time for Friedel-Crafts reaction, and the post-treatment process are consistent with the setting(s) in the method for preparing the compound having the structure represented by formula I under the condition that R₅—X— is a hydroxyl group as described above, and they will not be repeated here.

In the present disclosure, after the compound having the structure represented by formula II is obtained, the compound having the structure represented by formula II, R₅—X—H, and the catalyst II are mixed to undergo a condensation reaction to obtain a compound having the structure represented by formula I.

In the present disclosure, R₅—X— in R₅—X—H is the same as R₅—X— in the structure represented by formula I above, and it will not be repeated here. In some embodiments, the catalyst II is a mixture of 2-(7-azabenzotriazole-yl)-N,N,N′,N′-tetramethyluronium hexafluorophosphate (HATU) and N,N-diisopropylethylamine (DIPEA). In some embodiments, a molar ratio of the 2-(7-azabenzotriazole-yl)-N,N,N′,N′-tetramethyluronium hexafluorophosphate to the N,N-diisopropylethylamine is in the rang of 1.1:(4-10), and preferably 1.1:6.

In some embodiments of the present disclosure, a molar ratio of the compound having the structure represented by formula II, R₅—X—H, and the catalyst II is in the range of 1:1.1:(3-7.1), and preferably 1:1.1:3.

In some embodiments of the present disclosure, the mixing is performed in dimethylformamide (DMF). In some embodiments of the present disclosure, a molar ratio of the compound having the structure represented by formula II to the dimethylformamide is in the range of 1:(1-1.1), and preferably 1:1.1.

In some embodiments of the present disclosure, the compound having the structure represented by formula II, R₅—X—H and the catalyst II are added in sequence and mixed, which helps to control the reaction temperature and ensure the complete progress of the condensation reaction. In some embodiments of the present disclosure, the mixing is carried out under a stirring condition. In some embodiments, the stirring is performed at a stirring speed of 160-180 r/min, and preferably 180 r/min.

In some embodiments of the present disclosure, the condensation reaction is performed at room temperature. The room temperature herein refers to 25° C. In some embodiments of the present disclosure, the progress of the condensation reaction is tracked by TLC to determine the end time of the reaction. In some embodiments of the present disclosure, the condensation reaction is performed for 2-5 h, and preferably 2 h. The time for condensation reaction is specifically started counting after the completion of the addition of the R₅—X.

In some embodiments of the present disclosure, after the condensation reaction, the system obtained from the condensation reaction is purified by column chromatography to obtain the compound having the structure represented by formula I. In the present disclosure, there is no special limitations on the column chromatography, and column chromatography well known to those skilled in the art may be used. In a specific embodiment of the present disclosure, the mobile phase for the column chromatography is a mixed solution of cyclohexane and ethyl acetate. In some embodiment, a molar ratio of cyclohexane to ethyl acetate is in the range of 100 (7-10).

The present disclosure also provides a disinfectant, an active ingredient of which includes the α-substituted phenyl structure-containing compound described in the above technical solutions. In some embodiments of the present disclosure, the disinfectant is prepared by a process including dissolving the α-substituted phenyl structure-containing compound in water to prepare a solution with a concentration of 0.1-15 g/L; or compounding the α-substituted phenyl structure-containing compound with other additives. The disinfectant according to the present disclosure has better sterilization and disinfection effects, and has broad application prospects in sterilization and disinfection products.

The technical solutions of the present disclosure will be clearly and completely described below in conjunction with the examples of the present disclosure. Obviously, the described examples are only a part of the examples of the present disclosure, rather than all the examples. Based on the examples of the present disclosure, all other examples obtained by those of ordinary skill in the art without creative labor shall fall within the scope of the present disclosure.

### Example 1

500 mg of catechol, 420 mg of cyclohexane and 0.485 mL of glyoxylic acid monohydrate were sequentially added to a reaction flask. With 10 mL of water as the solvent, in the presence of 769 mg of p-toluenesulfonic acid catalyst, the resulting mixture was stirred at a stirring speed of 160 r/min and reacted at 80° C. for 5 h. The reaction was monitored by TLC. After the reaction was completed, the reaction solution was cooled to room temperature, and the pH value of the reaction solution was adjusted to 2. The resulting mixture was subjected to an extraction with ethyl acetate, obtaining a crude product of 2-cyclohexyl-2-(3,4-dihydroxy phenyl)acetic acid. The crude product was then recrystallized with ethanol, obtaining 1 g of 2-cyclohexyl-2-(3,4-dihydroxyphenyl)acetic acid.

The 2-cyclohexyl-2-(3,4-dihydroxyphenyl)acetic acid was dissolved in 10 mL of DMF. 1.8 g of HATU, 3.3 mL of DIPEA and 1 mL of aqueous methylamine solution with a mass percentage of 40% were added thereto in sequence. The resulting mixture was stirred at a stirring speed of 180 r/min and reacted at 25° C. for 2 h. The reaction was monitored by TLC. After the reaction was completed, the reaction solution was purified by column chromatography (with a mobile phase of cyclohexane/ethyl acetate, and a molar ratio of cyclohexane to ethyl acetate of 100:7), obtaining 2-cyclohexyl-2-(3,4-dihydroxyphenyl)-N-methylacetamide, which had a structural formula of

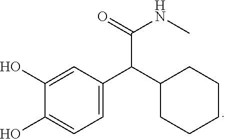 [SMILES: CNC(=O)C(c1ccc(O)c(O)c1)C1CCCCC1]

The 2-cyclohexyl-2-(3,4-dihydroxyphenyl)-N-methylacetamide was obtained as an earthy yellow solid, with a melting point of higher than 300° C., and a yield of 61%. Its analysis results are as follows:

¹H NMR (400 MHz, CDCl₃) δ 9.06 (s, 1H), 8.94 (s, 1H), 7.26 (q, J=3.7 Hz, 1H), 6.79 (d, J=1.0 Hz, 1H), 6.76 (d, J=0.9 Hz, 2H), 3.72 (d, J=7.1, 1.1 Hz, 1H), 2.75 (d, J=3.7 Hz, 3H), 2.37 (h, J=7.0 Hz, 1H), 1.64-1.54 (m, 4H), 1.53-1.49 (m, 2H), 1.49-1.43 (m, 4H).

¹³C NMR (100 MHz, CDCl₃) δ 173.98, 145.55, 144.52, 132.88, 121.97, 116.51, 115.87, 58.78, 39.82, 28.65, 26.23, 25.91, 25.89.

### Example 2

500 mg of 4-hydroxybenzene, 456 mg of benzene and 0.568 mL of glyoxylic acid monohydrate were sequentially added to a reaction flask. With 10 mL of water as the solvent, in the presence of 300 mg of solid strong acid catalyst, the resulting mixture was stirred at a stirring speed of 160 r/min and reacted at 70° C. for 5 h. The reaction was monitored by TLC. After the reaction was completed, the reaction solution was cooled to room temperature, and the pH value of the reaction solution was adjusted to 2. The resulting mixture was subjected to an extraction with ethyl acetate, obtaining a crude product of 2-(4-hydroxyphenyl)-N-isopropyl-2-phenylacetic acid. The crude product was then recrystallized with ethanol, obtaining 1.1 g of 2-(4-hydroxyphenyl)-N-isopropyl-2-phenylacetic acid.

The 2-(4-hydroxyphenyl)-N-isopropyl-2-phenylacetic acid was dissolved in 20 mL of DMF. 2.2 g of HATU, 3.9 mL of DIPEA, and 1.1 mL of isopropylamine were added thereto in sequence. The resulting mixture was stirred at a stirring speed of 180 r/min and reacted at 25° C. for 2 h. The reaction was monitored by TLC. After the reaction was completed, the reaction solution was purified by column chromatography (with a mobile phase of cyclohexane/ethyl acetate, and a molar ratio of cyclohexane to ethyl acetate of 100:7), obtaining 2-(4-hydroxyphenyl)-N-isopropyl-2-phenylacetamide, which had a structural formula of

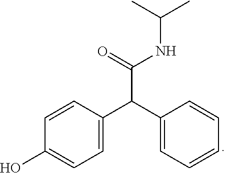 [SMILES: CC(C)NC(=O)C(c1ccccc1)c1ccc(O)cc1]

The 2-(4-hydroxyphenyl)-N-isopropyl-2-phenylacetamide was obtained as an earthy yellow solid, with a melting point of higher than 300° C., and a yield of 67%. Its analysis results are as follows:

¹H NMR (400 MHz, CDCl₃) δ 7.84 (s, 1H), 7.54 (d, J=7.3 Hz, 1H), 7.28 (d, J=2.1 Hz, 3H), 7.27-7.23 (m, 2H), 7.21 (t, J=1.0 Hz, 2H), 6.72-6.69 (m, 2H), 5.17 (s, J=0.8 Hz, 1H), 3.96 (dq, J=13.7, 6.9 Hz, 1H), 1.22 (d, J=6.8 Hz, 3H), 1.17 (d, J=6.8 Hz, 3H).

¹³C NMR (100 MHz, CDCl₃) δ 173.60, 156.72, 138.18, 132.74, 129.33, 129.17, 128.47, 127.71, 115.40, 58.62, 44.34, 22.81.

### Example 3

500 mg of 3,4-difluorobenzene, 292 mg of benzene and 0.363 mL of glyoxylic acid monohydrate were sequentially added to a reaction flask. With 10 mL of water as the solvent, in the presence of 260 mg of solid strong acid catalyst, the resulting mixture was stirred at a stirring speed of 180 r/min and reacted at 70° C. for 5 h. The reaction was monitored by TLC. After the reaction was completed, the reaction solution was cooled to room temperature, and the pH value of the reaction solution was adjusted to 2. The resulting mixture was subjected to an extraction with ethyl acetate, obtaining a crude product of 2-(3,4-difluorophenyl)-N-methyl-2-phenylacetic acid. The crude product was recrystallized with ethanol, obtaining 800 mg of 2-(3,4-difluorophenyl)-N-methyl-2-phenylacetic acid.

The 2-(3,4-difluorophenyl)-N-methyl-2-phenylacetic acid was dissolved in 10 mL of DMF. 1.3 g of HATU, 2.3 mL of DIPEA and 1 mL of aqueous methylamine solution with a mass percentage of 40% were added thereto in sequence. The resulting mixture was stirred at a stirring speed of 180 r/min and reacted at 25° C. for 2 h. The reaction was monitored by TLC. After the reaction was completed, the reaction solution was purified by column chromatography (with a mobile phase of cyclohexane/ethyl acetate, and a molar ratio of cyclohexane to ethyl acetate of 10:1), obtaining 2-(3,4-difluorophenyl)-N-methyl-2-phenylacetamide, which had a structural formula of

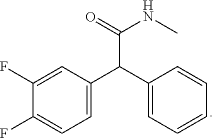 [SMILES: CNC(=O)C(c1ccccc1)c1ccc(F)c(F)c1]

The 2-(3,4-difluorophenyl)-N-methyl-2-phenylacetamide was obtained as an earthy yellow solid, with a melting point of higher than 300° C., and a yield of 79%. Its analysis results are as follows:

¹H NMR (400 MHz, CDCl₃) δ 7.28 (d, J=3.3 Hz, 3H), 7.27-7.25 (m, 2H), 7.25-7.22 (m, 1H), 7.22-7.19 (m, 2H), 4.99 (s, 1H), 2.77 (d, J=3.5 Hz, 3H).

¹³C NMR (100 MHz, CDCl₃) δ 175.61, 151.84, 150.28, 138.03, 135.92, 129.17, 128.47, 127.75, 125.85, 117.39, 116.99, 55.40, 25.91.

### Example 4

500 mg of 4-hydroxybenzene, 538 mg of toluene and 0.568 mL of glyoxylic acid monohydrate were sequentially added to a reaction flask. With 20 mL of water as the solvent, in the presence of 240 mg of solid strong acid catalyst, the resulting mixture was stirred at a stirring speed of 180 r/min and reacted at 100° C. for 7 h. The reaction was monitored by TLC. After the reaction was completed, the reaction solution was cooled to room temperature, diluted with 20 mL of ethyl acetate and filtered. The filtrate was concentrated in vacuum, and the solid obtained from the filtration was dissolved in diethyl ether. The resulting solution was subjected to an extraction for three times with 1.0 mol/L aqueous sodium carbonate solution, each time with 15 mL. The aqueous layer obtained from the extraction was acidified with concentrated hydrochloric acid to a pH value of 2, and then filtrated. The solid obtained after the filtration was collected, obtaining 2-(4-hydroxyphenyl)-2-p-tolueneacetic acid, which has a structural formula of

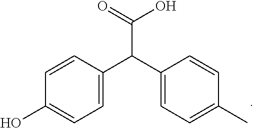 [SMILES: Cc1ccc(C(C(=O)O)c2ccc(O)cc2)cc1]

The 2-(4-hydroxyphenyl)-2-p-tolueneacetic acid was obtained as a solid, with a yield of 84%. Its analysis results are as follows:

¹H NMR (400 MHz, CDCl₃) δ 7.82 (s, 1H), 7.29-7.25 (m, 2H), 7.23-7.19 (m, 2H), 7.19-7.16 (m, 2H), 6.73-6.69 (m, 2H), 5.04 (s, J=0.9 Hz, 1H), 2.35 (s, J=1.0 Hz, 3H).

¹³C NMR (100 MHz, CDCl₃) δ 178.06, 156.74, 137.83, 136.16, 131.64, 129.42, 129.08, 128.75, 115.43, 58.72, 20.98.

### Example 5

500 mg of 3,4-dihydroxybenzene, 1.1 mol of furan and 1.3 mL of glyoxylic acid monohydrate were sequentially added to a reaction flask. With 20 mL of water as the solvent, in the presence of 200 mg of solid strong acid catalyst and catalytic amount of p-toluenesulfonic acid, the resulting mixture was stirred at a stirring speed of 180 r/min and reacted at 70° C. for 5 h. The reaction was monitored by TLC. After the reaction was completed, the reaction solution was cooled to room temperature, and the pH value of the reaction solution was adjusted to 2. The resulting mixture was subjected to an extraction with ethyl acetate, obtaining a crude product of 2-(3,4-dihydroxyphenyl)-2-(2-furyl)-N-methylacetic acid. The crude product was recrystallized with ethanol, obtaining 860 mg g of 2-(3,4-dihydroxyphenyl)-2-(2-furyl)-N-methylacetic acid.

The 2-(3,4-dihydroxyphenyl)-2-(2-furyl)-N-methylacetic acid was dissolved in 10 mL of DMF. 1.6 g of HATU, 2.3 mL of DIPEA and 1.2 mL of aqueous methylamine solution with a mass percentage of 40% were added thereto in sequence. The resulting mixture was stirred at a stirring speed of 180 r/min and reacted at 25° C. for 2 h. The reaction was monitored by TLC. After the reaction was completed, the reaction solution was purified by column chromatography (with a mobile phase of cyclohexane/ethyl acetate, and a molar ratio of cyclohexane to ethyl acetate of 10:1), obtaining 2-(3,4-dihydroxyphenyl)-2-(2-furyl)-N-methylacetamide, which had a structural formula of

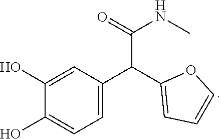 [SMILES: CNC(=O)C(c1ccc(O)c(O)c1)c1ccco1]

The 2-(3,4-dihydroxyphenyl)-2-(2-furyl)-N-methylacetamide was obtained as an earthy yellow solid, with a melting point of higher than 300° C., and a yield of 77%. Its analysis results are as follows:

¹H NMR (400 MHz, CDCl₃) δ 9.15 (s, 1H), 8.96 (s, 1H), 7.42 (dd, J=7.4, 1.5 Hz, 1H), 7.30 (q, J=3.5 Hz, 1H), 6.87-6.83 (m, 2H), 6.78 (dt, J=7.6, 0.8 Hz, 1H), 6.34 (t, J=7.4 Hz, 1H), 6.28 (dd, J=7.5, 1.6 Hz, 1H), 5.26 (s, J=0.9 Hz, 1H), 2.79 (s, J=3.5 Hz, 3H).

¹³CNMR (100 MHz, CDCl₃) δ 171.95, 151.14, 145.42, 145.33, 142.18, 128.50, 120.85, 115.90, 115.43, 110.66, 109.95, 55.54, 25.91.

### Example 6

500 mg of 3,4-dihydroxybenzene, 395 mol of pyridine and 0.485 mL of glyoxylic acid monohydrate were sequentially added to a reaction flask. With 10 mL of water as the solvent, in the presence of 200 mg of solid strong acid catalyst, the resulting mixture was stirred at a stirring speed of 180 r/min and reacted at 70° C. for 5 h, and the reaction was monitored by TLC. After the reaction was completed, the reaction solution was cooled to room temperature, and the pH value of the reaction solution was adjusted to 2. The resulting mixture was subjected to an extraction with ethyl acetate, obtaining a crude product of 2-(3,4-dihydroxyphenyl)-N-methyl-2-(4-pyridyl) acetic acid. The crude product was then recrystallized with ethanol, obtaining 800 mg g of 2-(3,4-dihydroxyphenyl)-N-methyl-2-(4-pyridyl)acetic acid.

The 2-(3,4-dihydroxyphenyl)-N-methyl-2-(4-pyridyl)acetic acid was dissolved in 10 mL of DMF. 1.4 g of HATU, 2.7 mL of DIPEA, and 1.2 mL of aqueous methylamine solution with a mass percentage of 40% were added thereto in sequence. The resulting mixture was stirred at a stirring speed of 180 r/min and reacted at 25° C. for 2 h. The reaction was monitored by TLC. After the reaction was completed, the reaction solution was purified by column chromatography (with a mobile phase of cyclohexane/ethyl acetate, and a molar ratio of cyclohexane to ethyl acetate of 10:1), obtaining 2-(3,4-dihydroxyphenyl)-N-methyl-2-(4-pyridyl)acetamide, which had a structural formula of

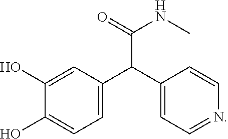 [SMILES: CNC(=O)C(c1ccncc1)c1ccc(O)c(O)c1]

The 2-(3,4-dihydroxyphenyl)-N-methyl-2-(4-pyridyl)acetamide was obtained as an earthy yellow solid, with a melting point of higher than 300° C., and a yield of 60%. Its analysis results are as follows:

¹H NMR (400 MHz, DMSO) δ 9.48 (s, 2H), 7.49 (s, 1H), 8.54-8.43 (m, 2H), 7.27-7.01 (m, 2H), 6.83 (d, 1H), 6.81 (s, 1H), 6.62 (d, 1H), 5.02 (s, 1H), 2.83 (s, 3H).

### Example 7

500 mg of benzene and 0.684 mL of glyoxylic acid monohydrate were sequentially added to a reaction flask. With 20 mL of water as the solvent, in the presence of 320 mg of solid strong acid catalyst, the resulting mixture was stirred at a stirring speed of 180 r/min and reacted at 80° C. for 7 h. The reaction was monitored by TLC. After the reaction was completed, the reaction solution was cooled to room temperature, diluted with 20 mL of ethyl acetate and filtered. The filtrate was concentrated in vacuum, and the solid obtained from the filtration was dissolved in diethyl ether. The resulting solution was subjected to an extraction for three times with 1.0 mol/L aqueous sodium carbonate solution, each time with 15 mL. The aqueous layer was acidified with concentrated hydrochloric acid to a pH value of 2, and then filtrated. The solid obtained after a filtration was collected, obtaining 2,2-diphenylacetic acid, which had a structural formula of

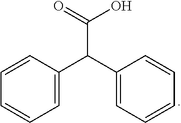 [SMILES: O=C(O)C(c1ccccc1)c1ccccc1]

The 2,2-diphenylacetic acid was obtained as a solid powder, with a yield of 83%. Its analysis results are as follows:

¹H NMR (400 MHz, DMSO) δ 12.02 (s, 1H), 7.37-7.21 (m, 10H), 4.93 (s, 1H).

### Example 8

500 mg of catechol and 0.485 mL of glyoxylic acid monohydrate were sequentially added to a reaction flask. With 20 mL of water as the solvent, in the presence of 330 mg of solid strong acid catalyst, the resulting mixture was stirred at a stirring speed of 180 r/min and reacted at 80° C. for 5 h. The reaction was monitored by TLC. After the reaction was completed, the reaction solution was cooled to room temperature, and the pH value of the reaction solution was adjusted to 2. The resulting mixture was subjected to an extraction with ethyl acetate, obtaining a crude product of 2,2-di-(3,4-dihydroxyphenyl)acetic acid, which had a structural formula of

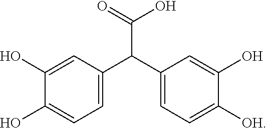 [SMILES: O=C(O)C(c1ccc(O)c(O)c1)c1ccc(O)c(O)c1]

The 2,2-bis-(3,4-dihydroxyphenyl)acetic acid was obtained as a light yellow solid powder with a yield of 90%. Its analysis results are as follows:

¹H NMR (400 MHz, DMSO) δ 12.07 (s, 1H), 9.50 (s, 4H), 6.83-6.61 (m, 4H), 4.91 (s, 1H).

### Test Example 1

(1) Preparation of the Sample to be Tested:

4-chloro-3,5-dimethylphenol was dissolved in an aqueous dimethyl sulfoxide (DMSO) solution with a mass percentage of 1%, and then diluted with sterilized ultrapure water to 4-chloro-3,5-dimethylphenol solutions with concentrations of 0.31 g/L and 0.15 g/L, respectively.

Phenol was dissolved with sterilized ultrapure water and diluted to phenol solutions with concentrations of 6.25 g/L, 3.12 g/L, 1.56 g/L, 0.78 g/L, 0.31 g/L and 0.15 g/L, respectively.

2-(3,4-dihydroxyphenyl)-2-(2-furyl)-N-methylacetamide (drug to be tested) prepared in Example 5 was dissolved in sterilized ultrapure water and diluted to drug solutions with concentrations of 6.25 g/L, 3.12 g/L, 1.56 g/L, 0.78 g/L, 0.31 g/L and 0.15 g/L, respectively.

(2) Preparation of a Neutralizer:

a. 5 mL of Tween-80 and 0.2 g of lecithin were heated to dissolve.

b. 0.2 g of histidine was dissolved in 5 mL of purified water and heated in a water bath at 54° C. to dissolve.

c. The dissolved Tween-80 and lecithin were added to the cooled histidine solution, mixed to 100 mL, and the resulting mixture was subjected to an autoclaving.

(3) Formula of 0.03 mol/L PBS buffer: 3.36 g of PBS was weighed and dissolved in 100 mL of purified water, and subjected to a sterilization treatment.

(4) Experimental Method for Selecting a Neutralizer:

Escherichia coli ATCC 25922 and Staphylococcus aureus ATCC 29213 were used as the experimental strains, and the concentration of the drug to be tested was set to be 2.5 g/L. The experiment was carried out according to the suspension quantitative sterilization procedure, with three repetitions, and six parallel groups:

Group I: 100 μL of a bacterial suspension (Escherichia coli ATCC 25922 with a concentration of 1×10⁸ CFU/mL and Staphylococcus aureus ATCC 29213 with a concentration of 1×10⁸ CFU/mL) interacted with 400 μL of the drug to be tested for 5 min; 50 μL of the mixed solution and 450 μL of PBS buffer were mixed to be uniform and diluted; the viable bacteria therein was counted;

Group II: 100 μL a bacterial suspension (Escherichia coli ATCC 25922 with a concentration of 1×10⁸ CFU/mL and Staphylococcus aureus ATCC 29213 with a concentration of 1×10⁸ CFU/mL) interacted with 400 μL of the drug to be tested for 5 min; 50 μL of the mixed solution and 450 μL of a neutralizer were mixed to be uniform and reacted for 10 min, and diluted; the viable bacteria therein was counted;

Group III: 10 μL of a bacterial suspension, 40 μL of sterile water, and 450 μL of a neutralizer were reacted for 10 min; the reaction solution was diluted, and the viable bacteria therein was counted;

Group IV: 40 μL of 2.5 g/L drug solution to be tested was reacted with 450 μL of a neutralizer for 10 min, and 10 μL of a bacterial suspension was then added thereto; the reaction solution was diluted, and the viable bacteria therein was counted;

Group V: 100 μL of bacterial suspension and 400 μL of a neutralizer were reacted for 5 min; 50 μL of the mixture and 450 μL of PBS buffer were mixed to be uniform, and then diluted; the viable bacteria therein was counted;

Group VI: a mixture of culture medium and PBS buffer was used as negative control.

Determination of the Results:

Group I had no bacteria growth or a small amount of bacteria growth;

Group II had bacteria growth, and the number of bacteria was not less than 100 CFU/mL;

the error rate of the number of bacteria between the Group III, Group IV, and Group V was less than or equal to 15%;

Group VI had no bacteria growth.

The results show that the neutralizer and its concentration are appropriate.

(5) Operation Method of Suspension Quantitative Experiment:

a. The freeze-dried strain tube was provided, opened up under an aseptic condition, and an appropriate amount of nutrient broth was added thereto with a capillary pipette. The strain was melted and dispersed by gently blowing and sucking several times. A test tube containing 5.0 mL-10.0 mL of nutrient broth medium was provided, and a small amount of bacterial suspension was dropped thereto, and incubated at 37° C. for 18 h-24 h. The bacterial suspension of the first generation culture was taken by using an inoculation loop, and streaked and inoculated onto a nutrient agar medium plate, and incubated at 37° C. for 18 h-24 h. The typical colonies from the second generation culture was picked out, inoculated onto a nutrient agar slant, and incubated at 37° C. for 18 h-24 h, obtaining the third generation culture.

b. The monoclonal strains of the third generation culture of Escherichia coli ATCC 25922, Staphylococcus aureus ATCC 29213, and Pseudomonas aeruginosa ATCC 27853 were picked out respectively and placed in 3 mL of NMB. The strains were shaken in a constant temperature culture shaker for 3 h with a rotation speed of 220 rpm, obtaining a bacterial suspension with a concentration of (1-5)×10⁸ CFU/mL.

c. 0.5 mL of bacterial suspension was added to a sterile test tube, and 0.5 mL of an organic interfering substance (0.03 mol/L PBS buffer) was then added thereto. They were mixed to be uniform, and placed in a water bath at 20° C.±1° C. for 5 min. 4.0 mL of the sample to be tested prepared in step (1) was drawn with a sterile pipette and injected into the mixed solution, mixed quickly, and the time was recorded immediately.

d. After the experimental bacteria interacted with the sample to be tested for the predetermined time of 1 min, 5 min, 15 min and 30 min. 0.5 mL of the mixture of the experimental bacteria and the sample to be tested was drawn respectively and added to 4.5 mL of a neutralizer, and mixed to be uniform.

e. After 10 min of neutralization, 1.0 mL of the sample solution was drawn, and the number of viable bacteria was determined by viable count method. After the viable count method, sample solution of each pipette was inoculated onto 2 plates. When the number of colonies growing on the plate was large, 10-fold dilution was performed, and then the viable bacteria was cultured and counted.

f. At the same time, PBS buffer was used to replace the sample to be tested, and a parallel test was performed as a positive control.

g. All test samples were placed in a 37° C. incubator overnight, and the results were observed.

h. The experiment was repeated for 3 times, the concentration of viable bacteria (CFU/mL) of each group was calculated, and converted to logarithmic value (N), and then the killing logarithmic value was calculated according to the following equation:

Killing logarithmic value (KL)=the logarithmic value of the average viable bacteria concentration of the control group (No)−the logarithmic value of the viable bacteria concentration of the test group (Nx).

When calculating the killing logarithmic value, two digits after the decimal point was kept, and digital round-off was allowed. The test results are shown in Tables 1-4:

TABLE 1
Identification results of the drug to be tested on the neutralizer of
Escherichia coli and Staphylococcus aureus

| | Escherichia coli | Staphylococcus aureus |
|---|---|---|
| | ATCC 25922 | ATCC29213 |
| Group | (CFU/mL) | (CFU/mL) |
| I | 2 × 10⁴ | 3 × 10⁴ |
| II | 400 | 5 × 10⁴ |
| III | 3 × 10⁵ | 9 × 10⁵ |
| IV | 6 × 10⁶ | 7 × 10⁵ |
| V | 2 × 10⁵ | 6 × 10⁶ |
| VI | 0 | 0 |

TABLE 2
The bactericidal effect of the sample to be tested on Escherichia coli
ATCC 25922 (logarithmic value)

| | | Killing logarithmic | | | |
|---|---|---|---|---|---|
| | | value | | | |
| | Concentration | 1 | 5 | 15 | 30 |
| Sample to be tested | (g/L) | min | min | min | min |
| 4-Chloro-3,5-dimethylphenol | 0.31 | 6.17 | 6.17 | 6.17 | 6.17 |
| solution | 0.15 | 1.69 | 1.78 | 2.32 | 2.99 |
| Phenol solution | 6.25 | 3.57 | 3.69 | 5.86 | 6.17 |
| | 3.12 | 0.69 | 0.76 | 0.81 | 1.02 |
| | 1.56 | 0.47 | 0.65 | 0.78 | 0.99 |
| | 0.78 | 0.32 | 0.46 | 0.63 | 0.79 |
| | 0.31 | 0.29 | 0.31 | 0.46 | 0.58 |
| | 0.15 | 0.21 | 0.29 | 0.43 | 0.47 |
| Drug solution to be tested | 6.25 | 6.17 | 6.17 | 6.17 | 6.17 |
| | 3.12 | 4.78 | 6.17 | 6.17 | 6.17 |
| | 1.56 | 3.02 | 3.67 | 4.12 | 4.56 |
| | 0.78 | 1.59 | 1.78 | 2.09 | 2.21 |
| | 0.31 | 1.56 | 1.64 | 1.87 | 1.99 |
| | 0.15 | 0.56 | 0.89 | 1.43 | 1.51 |

TABLE 3
The bactericidal effect of the sample to be tested on Staphylococcus
aureus ATCC 29213 (logarithmic value)

| | | Killing logarithmic | | | |
|---|---|---|---|---|---|
| | | value | | | |
| | Concentration | 1 | 5 | 15 | 30 |
| Sample to be tested | (g/L) | min | min | min | min |
| 4-Chloro-3,5-dimethylphenol | 0.31 | 3.12 | 3.30 | 3.45 | 4.13 |
| solution | 0.15 | 2.07 | 2.74 | 2.90 | 3.16 |
| Phenol solution | 6.25 | 2.52 | 2.62 | 2.74 | 2.99 |
| | 3.12 | 1.13 | 1.21 | 1.23 | 1.26 |
| | 1.56 | 0.86 | 0.91 | 0.99 | 1.02 |
| | 0.78 | 0.79 | 0.82 | 0.89 | 0.95 |
| | 0.31 | 0.67 | 0.69 | 0.73 | 0.86 |
| | 0.15 | 0.45 | 0.48 | 0.55 | 0.73 |
| Drug solution to be tested | 6.25 | 3.99 | 4.47 | 5.77 | 6.66 |
| | 3.12 | 1.77 | 1.92 | 1.97 | 2.02 |
| | 1.56 | 1.54 | 1.68 | 1.84 | 1.92 |
| | 0.78 | 1.21 | 1.32 | 1.42 | 1.48 |
| | 0.31 | 0.99 | 1.03 | 1.16 | 1.32 |
| | 0.15 | 0.86 | 0.91 | 1.03 | 1.09 |

TABLE 4
The bactericidal effect of the sample to be tested on Pseudomonas
aeruginosa ATCC 27853 (logarithmic value)

| | | Killing logarithmic | | | |
|---|---|---|---|---|---|
| | | value | | | |
| | Concentration | 1 | 5 | 15 | 30 |
| Sample to be tested | (g/L) | min | min | min | min |
| 4-Chloro-3,5-dimethylphenol | 0.31 | 4.77 | 4.87 | 4.91 | 4.95 |
| solution | 0.15 | 0.78 | 0.87 | 0.94 | 0.96 |
| Phenol solution | 6.25 | 3.07 | 3.65 | 4.55 | 5.25 |
| | 3.12 | 1.56 | 1.86 | 1.92 | 1.98 |
| | 1.56 | 1.21 | 1.25 | 1.34 | 1.65 |
| | 0.78 | 0.99 | 1.08 | 1.12 | 1.16 |
| | 0.31 | 0.76 | 0.79 | 0.84 | 0.93 |
| | 0.15 | 0.46 | 0.51 | 0.57 | 0.67 |
| drug solution to be tested | 6.25 | 5.25 | 5.25 | 5.25 | 5.25 |
| | 3.12 | 3.07 | 3.71 | 5.25 | 5.25 |
| | 1.56 | 1.25 | 1.27 | 1.29 | 1.31 |
| | 0.78 | 0.97 | 0.99 | 1.07 | 1.10 |
| | 0.31 | 0.86 | 0.89 | 0.92 | 0.95 |
| | 0.15 | 0.75 | 0.78 | 0.84 | 0.88 |

From the test results in Tables 2-4, it can be seen that when the concentration is 3.125 g/L, the killing logarithmic value against Escherichia coli ATCC 25922 within 1 min is more than or equal to 5.00, and the killing logarithmic value against Pseudomonas aeruginosa ATCC 27853 within 15 min is more than or equal to 5.00; when the concentration is 6.25 g/L, the killing logarithmic value against Staphylococcus aureus ATCC 29213 within 15 min is more than or equal to 5.00. Therefore, it meets the sanitary requirements of phenolic disinfectants according to GB27947-2011, and has excellent sterilization effects.

### Test Example 2

Test was performed according to the sanitary requirements of medical device disinfectants GB/T27949-2011.

(1) Preparation of the Sample to be Tested:

o-Phthalaldehyde was dissolved in sterilized ultrapure water and diluted into o-phthalaldehyde solutions with concentrations of 5 g/L, 10 g/L, and 15 g/L, respectively.

2-(4-hydroxyphenyl)-2-p-tolueneacetic acid (drug to be tested) prepared in Example 4 was dissolved in sterilized ultrapure water and diluted to drug solutions to be tested with concentrations of 5 g/L, 10 g/L, and 15 g/L, respectively.

(2) Preparation of a Neutralizer:

a. 5 mL of Tween-80 and 0.2 g of lecithin were heated to dissolve.

b. 0.2 g of histidine was dissolved in 5 mL of purified water and heated in a water bath at 54° C. to dissolve.

c. The dissolved Tween-80 and lecithin were added to the cooled histidine solution, mixed to 100 mL, and the resulting mixture was subjected to an autoclaving.

(3) Formula of 0.03 mol/L PBS buffer: 3.36 g of PBS was weighed and dissolved in 100 mL of purified water, and subjected to a sterilization treatment.

(4) Operation Method of Suspension Quantitative Experiment:

a. The Bacillus subtilis var. niger spores ATCC 9372 freeze-dried strain tube was provided, opened up under an aseptic condition, and an appropriate amount of nutrient broth was added thereto with a capillary pipette. The strain was melted and dispersed by gently blowing and sucking several times. A test tube containing 5.0 mL-10.0 mL of nutrient broth medium was provided, and a small amount of bacterial suspension was dropped thereto, and incubated at 37° C. for 18 h-24 h. The bacterial suspension of the first generation culture was taken by using an inoculation loop.

b. The monoclonal strains of the first generation culture were picked out and placed in 3 mL of NMB. The strains were shaken in a constant temperature culture shaker for 3 h with a rotation speed of 220 rpm, obtaining a bacterial suspension with a concentration of (1-5)×10⁸ CFU/mL.

c. 0.5 mL of bacterial suspension was added to a sterile test tube, and 0.5 mL of an organic interfering substance (0.03 mol/L PBS buffer) was then added thereto. They were mixed to be uniform, and placed in a water bath at 20° C.±1° C. for 5 min. 4.0 mL of the sample to be tested prepared in step (1) was drawn with a sterile pipette and injected into the mixed solution, mixed quickly, and the time was recorded immediately.

d. After the experimental bacteria interacted with the sample to be tested for the predetermined time. 0.5 mL of the mixture of the experimental bacteria and a disinfectant was drawn respectively and added to 4.5 mL of a neutralizer, and mixed to be uniform.

e. After 10 min of neutralization, 1.0 mL of the sample solution was drawn, and the number of viable bacteria was determined by viable count method. After the viable count method, sample solution of each pipette was inoculated onto 2 plates. When the number of colonies growing on the plate was large, 10-fold dilution was performed, and then the viable bacteria was cultured and counted.

h. At the same time, PBS buffer was used to replace the sample to be tested, and a parallel test was performed as a positive control.

f. All test samples were placed in a 37° C. incubator overnight, and the results were observed.

g. The experiment was repeated for 3 times, the concentration of viable bacteria (CFU/mL) of each group was calculated, and converted to logarithmic value (N), and then the killing logarithmic value was calculated according to the following equation:

Killing logarithmic value (KL)=the logarithmic value of the average viable bacteria concentration of the control group (No)−the logarithmic value of the viable bacteria concentration of the test group (Nx).

When calculating the killing logarithmic value, two digits after the decimal point was kept, and digital round-off was allowed.

(5) Operation Method of the Carrier Immersion Quantitative Sterilization Test:

a. A sterile small plate was provided, and marked with the concentration of the sample to be tested injected. The sample to be tested of the corresponding concentration was drawn and injected into the plate in an amount of 5.0 mL per piece.

b. 3 pieces of Bacillus subtilis var. niger spores were placed on the plate by using a sterile tweezer, and soaked in the sample to be tested.

c. After the bacteria and drugs interacted for each predetermined time, the bacteria pieces were taken out by using a sterile tweezer and transferred into a test tube containing 5.0 mL of a neutralizer. The test tube was vibrated 80 times in the palm of the hand to wash the bacteria on the bacteria piece into the neutralization solution, left to stand for another 10 min to fully neutralize. Finally after further mixing, 1.0 mL of the mixture was drawn and directly inoculated onto the plate, 2 plates for each pipette, and the number of viable bacteria was determined.

d. Another plate was provided, and 10.0 mL of PBS buffer was injected instead of the sample to be tested. 2 pieces of bacteria were added thereto as the positive control group, and the subsequent test steps and viable bacteria culture and counting were the same as the above test groups.

e. All test samples were cultured overnight in a 37° C. incubator, and the results were observed.

f. The experiment was repeated for 3 times (including the control group), and the number of viable bacteria (CFU/piece) in each group was calculated and converted to a logarithmic value (N).

(6) Determination Method of the Corrosion of Disinfectant to Metal:

Carbon steel, stainless steel, copper and aluminum were made into wafers with a diameter of 24.0±0.1 mm, a thickness of 1.0 mm, and having a small hole with a diameter of about 2.0 mm, and a total surface area of about 9.80 cm². Wafers were ground to remove the surface oxidation layer, washed and dried. The dried wafers were weighed, and measured for the diameter, pore size and thickness, as metal samples.

The metal samples were soaked into the sample to be tested. Each metal sample should be soaked in 200 mL of the sample to be tested, for 72 h at one time, 3 metal samples for each test, each metal sample at an interval of not less than 1 cm, which could be carried out in the same container (containing 600 mL of a disinfectant solution). After 72 h of soaking, the metal samples were taken out, first rinsed with tap water, then brushed by using a brush to remove corrosion products thereon. After removing corrosion products, the metal samples were washed, and water thereon was adsorbed with a coarse filter paper. The metal samples were then placed onto a petri dish with filter paper, and placed together with the petri dish in an oven at 50° C. for 1 h, and then picked up with a tweezer. When the metal samples were cooled to room temperature, the cooled metal samples were placed on a balance and weighed separately. It is necessary to wear clean gloves when weighing and before testing, and do not touch the metal samples directly with your hands.

The color changes of the metal samples were observed and recorded, and expressed as the average value of the metal corrosion rate (R). The weight loss value of the blank control sample should be subtracted during the calculation. The calculation was carried out according to the following equation:

$$R = \frac{8.76 \times {10}^{′} \times ( m - {m}_{t} - {m}_{k} )}{S \times t \times d}$$

where R represents the corrosion rate, in mm/a (millimeters/year); m represents the weight of the metal sample before the test, in g; mₜ represents the weight of the metal sample after the test, in g; mₖ represents the weight loss value of the metal sample after chemical treatment to remove corrosion products, in g (for those without chemical removal in the test, the mₖ value is deleted from the formula when calculating); S represents the total surface area of the metal sample, in cm²; t represents the test time, in h; d represents the density of the metal material, in kg/m³.

Corrosion classification standard

| Corrosion rate R (mm/a) | level |
|---|---|
| <0.0100 | basically no corrosion |
| 0.0100 to <0.100 | mild corrosion |
| 0.100 to <1.00 | moderate corrosion |
| ≥1.00 | severe corrosion |

The test results are shown in Tables 5-7.

TABLE 5
The killing logarithmic value of the sample to be tested against Bacillus
subtilis var. niger spores ATCC 9372 (suspension method)

| Sample to be | Concentration | Killing logarithmic value | | |
|---|---|---|---|---|
| tested | (g/L) | 10 min | 20 min | 30 min |
| Drug solution to | 5 | 5.01 | 5.12 | 5.34 |
| be tested | 10 | 5.11 | 5.35 | 5.39 |
| | 15 | 5.42 | 5.51 | 5.60 |
| o-Phthalaldehyde | 5 | 5.23 | 5.31 | 5.39 |
| solution | 10 | 5.41 | 5.49 | 5.57 |
| | 15 | 5.53 | 5.68 | 5.71 |

TABLE 6
The killing logarithmic value of the sample to be tested against Bacillus
subtilis var. niger sores ATCC 9372 (carrier method)

| Sample to be | Concentration | Killing logarithmic value | | | |
|---|---|---|---|---|---|
| tested | (g/L) | 1 h | 2h | 3 h | 4 h |
| Drug solution to | 5 | 0.92 | 1.06 | 1.21 | 1.33 |
| be tested | 10 | 1.13 | 1.30 | 1.91 | 2.05 |
| | 15 | 1.53 | 1.64 | 1.92 | 2.23 |
| o-Phthalaldehyde | 5 | 1.95 | 3.23 | 5.21 | 5.53 |
| solution | 10 | 2.19 | 5.12 | 5.26 | 5.53 |
| | 15 | 3.23 | 5.21 | 5.36 | 5.53 |

TABLE 7
Metal sheet corrosion test results

| | | Copper | Stainless | Carbon | Aluminum |
|---|---|---|---|---|---|
| Sample to be | Concentration | sheet | steel sheet | steel sheet | sheet |
| tested | (g/L) | (H62) | (304) | (A3) | (6061) |
| Drug solution to | 5 g/L | basically | basically | mild | mild |
| be tested | | no | no | corrosion | corrosion |
| | | corrosion | corrosion | | |
| | 10 g/L | basically | basically | moderate | mild |
| | | no | no | corrosion | corrosion |
| | | corrosion | corrosion | | |
| | 15 g/L | mild | basically | moderate | mild |
| | | corrosion | no | corrosion | corrosion |
| | | | corrosion | | |
| | 5 g/L | basically | basically | basically | basically |
| | | no | no | no | no |
| | | corrosion | corrosion | corrosion | corrosion |
| o-Phthalaldehyde | 10 g/L | basically | basically | basically | basically |
| solution | | no | no | no | no |
| | | corrosion | corrosion | corrosion | corrosion |
| | 15 g/L | basically | basically | basically | basically |
| | | no | no | no | no |
| | | corrosion | corrosion | corrosion | corrosion |

From the test results in Tables 5-7, it can be seen that the disinfectant according to the present disclosure has good water solubility, and has a solubility reaching 15 g/L. Given a concentration of 5 g/L, it has a killing logarithmic value against Bacillus subtilis var. niger spores within 10 min of not less than 5.00, and is non-corrosive to metals. Therefore, it is in line with the sanitary requirements of medical device disinfectants according to GB/T27949-2011.

### Test Example 3

According to the test method in Test Example 1, the disinfectants obtained in Examples 1-4 and Examples 6-8 were subjected to a sterilization test. The test results are shown in Tables 8-10.

TABLE 8
The killing logarithmic value against Escherichia coli ATCC 25922
(suspension method)

| Sample to be | Concentration | Action time | Activity (killing |
|---|---|---|---|
| tested | (g/L) | (min) | logarithmic value) |
| Example 1 | 6.25 | 30 | 1.23 |
| Example 2 | 6.25 | 30 | 0.97 |
| Example 3 | 6.25 | 30 | 1.14 |
| Example 4 | 6.25 | 30 | 0.78 |
| Example 6 | 6.25 | 30 | 1.64 |
| Example 7 | 6.25 | 30 | 1.02 |
| Example 8 | 6.25 | 30 | 1.73 |

TABLE 9
The killing logarithmic value against Staphylococcus aureus ATCC
29213 (suspension method)

| Sample to be | Concentration | Action time | Activity (killing |
|---|---|---|---|
| tested | (g/L) | (min) | logarithmic value) |
| Example 1 | 6.25 | 30 | 1.16 |
| Example 2 | 6.25 | 30 | 0.89 |
| Example 3 | 6.25 | 30 | 1.54 |
| Example 4 | 6.25 | 30 | 0.75 |
| Example 6 | 6.25 | 30 | 1.78 |
| Example 7 | 6.25 | 30 | 1.45 |
| Example 8 | 6.25 | 30 | 0.91 |

TABLE 10
The killing logarithmic value against Pseudomonas aeruginosa ATCC
27853 (suspension method)

| Sample to be | Concentration | Action time | Activity (killing |
|---|---|---|---|
| tested | (g/L) | (min) | logarithmic value) |
| Example 1 | 6.25 | 30 | 1.31 |
| Example 2 | 6.25 | 30 | 0.99 |
| Example 3 | 6.25 | 30 | 1.56 |
| Example 4 | 6.25 | 30 | 0.78 |
| Example 6 | 6.25 | 30 | 0.31 |
| Example 7 | 6.25 | 30 | 1.29 |
| Example 8 | 6.25 | 30 | 0.88 |

### Test Example 4

According to the test method in Test Example 2, the disinfectants obtained in Examples 1-3 and Examples 5-8 were subjected to a sterilization test. The test results are shown in Table 11.

TABLE 11
The killing logarithmic value against Bacillus subtilis var. niger spores
ATCC 9372 (suspension method)

| Sample to be | Concentration | Action time | Activity (killing |
|---|---|---|---|
| tested | (g/L) | (min) | logarithmic value) |
| Example 1 | 15 | 30 | 1.35 |
| Example 2 | 15 | 30 | 0.98 |
| Example 3 | 15 | 30 | 2.07 |
| Example 4 | 15 | 30 | 1.54 |
| Example 5 | 15 | 30 | 5.60 |
| Example 6 | 15 | 30 | 0.76 |
| Example 7 | 15 | 30 | 1.97 |
| Example 8 | 15 | 30 | 2.13 |

From the test results in Tables 8-11, it can be seen that the disinfectant according to the present disclosure has a good killing effect on pathogenic bacteria such as Escherichia coli, Staphylococcus aureus, Pseudomonas aeruginosa and Bacillus subtilis var. niger spores.

The description of the above embodiments is only used to help understand the method and the core idea of the present disclosure. It should be pointed out that for those of ordinary skill in the art, without departing from the principle of the present disclosure, several improvements and modifications could be made to the present disclosure, and these improvements and modifications also fall within the protection scope of the present disclosure. Various modifications to these embodiments are obvious to those skilled in the art, and the general principles defined herein could be implemented in other embodiments without departing from the spirit or scope of the present disclosure. Therefore, the present disclosure will not be limited to the embodiments shown in this document, but should conform to the widest scope consistent with the principles and novel features disclosed in this document.

What is claimed is:

1. An α-substituted phenyl structure-containing compound, which has a structure represented by formula I, 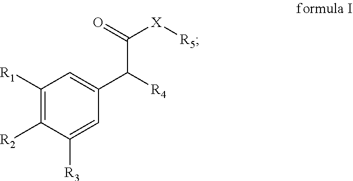
in formula I, each of R₁, R₂ and R₃ is independently selected from the group consisting of hydrogen, hydroxy, fluorine, and methoxy;
R₄ is selected from the group consisting of phenyl, 2-methylphenyl, 3-methylphenyl, 4-methylphenyl, 2-methoxyphenyl, 3-methoxyphenyl, 4-methoxyphenyl, 2-hydroxyphenyl, 3-hydroxyphenyl, 4-hydroxyphenyl, 2,3-dihydroxyphenyl, 2-bromophenyl, 3-bromophenyl, 4-bromophenyl, 2-fluorophenyl, 3-fluorophenyl, 4-fluorophenyl, 2,3-dimethylphenyl, 3,4-dimethylphenyl, 3,4,5-trimethylphenyl, 3,4,5-trimethoxyphenyl, 3,4,5-trihydroxyphenyl, pyridyl, 2-methylpyridyl, 3-methylpyridyl, cyclohexyl, furyl, and pyrrolyl;
R₅ is selected from the group consisting of hydrogen, methyl, ethyl, isopropyl, phenyl, and benzyl; and
X is selected from the group consisting of —CH₂—, —NH—, —O—, and —S—.

2. The compound as claimed in claim 1, wherein the α-substituted phenyl structure-containing compound is one selected from the group consisting of

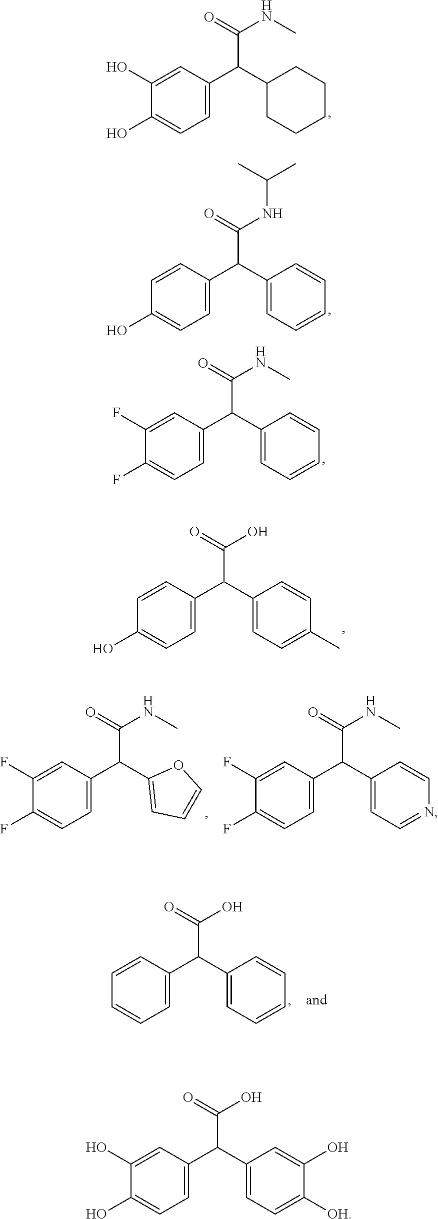

3. A method for preparing the α-substituted phenyl structure-containing compound as claimed in claim 1, wherein
under the condition that R₅—X— is hydroxyl group, the method for preparing the α-substituted phenyl structure-containing compound comprises
mixing 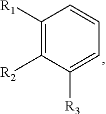
R₄—H, glyoxylic acid and a catalyst I to undergo a Friedel-Crafts reaction to obtain the compound having the structure represented by formula I;
under the condition that R₅—X— is a group other than hydroxyl, the method for preparing the α-substituted phenyl structure-containing compound comprises
mixing 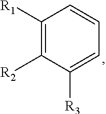
R₄—H, glyoxylic acid and a catalyst I to undergo a Friedel-Crafts reaction to obtain a compound having a structure represented by formula II; and
mixing the compound having the structure represented by formula II, R₅—X—H and a catalyst II to undergo a condensation reaction to obtain the compound having the structure represented by formula I;

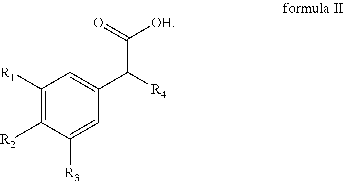

4. The method as claimed in claim 3, wherein a molar ratio of 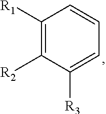
R₄—H and glyoxylic acid is in the rang of 1:(1.1-1.3):(1.3-1.5).

5. The method as claimed in claim 3, wherein the catalyst I is a strong acid, wherein the strong acid is sulfuric acid or nitric acid.

6. The method as claimed in claim 3, wherein the Friedel-Crafts reaction is performed at a temperature of 60-110° C. for 4-8 h.

7. The method as claimed in claim 3, wherein the catalyst II is a mixture of 2-(7-azabenzotriazole-1-yl)-N,N,N′,N′-tetramethyluronium hexafluorophosphate and N,N-diisopropylethylamine, and a molar ratio of the 2-(7-azabenzotriazole-1-yl)-N,N,N′,N′-tetramethyluronium hexafluorophosphate to the N,N-diisopropylethylamine is in the range of 1.1:(4-10).

8. The method as claimed in claim 7, wherein a molar ratio of the compound having the structure represented by formula II, R₅—X—H and the catalyst II is in the range of 1:1.1:(3-7.1).

9. The method as claimed in claim 3, wherein the condensation reaction is performed at ambient temperature for 2-5 h.

10. A disinfectant, an active ingredient of which comprises the α-substituted phenyl structure-containing compound as claimed in claim 1.

11. The method as claimed in claim 3, wherein the α-substituted phenyl structure-containing compound is one selected from the group consisting of

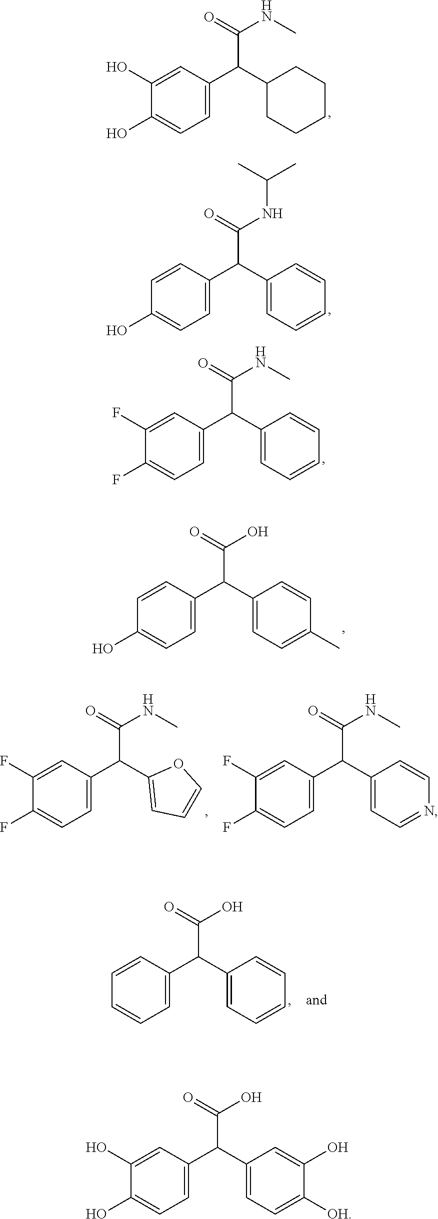

12. The disinfectant as claimed in claim 10, wherein the α-substituted phenyl structure-containing compound is one selected from the group consisting of [chemical structure]
